(19) United States
(12) Patent Application Publication
(10) Pub. No.: US 2015/0268157 A1
(43) Pub. Date: Sep. 24, 2015

(54) MULTILAYER NANOHOLE ARRAY SENSOR

(71) Applicant: Elwha LLC, Bellevue, WA (US)

(72) Inventors: Roderick A. Hyde, Redmond, WA (US); Lowell L. Wood,, JR., Bellevue, WA (US)

(21) Appl. No.: 14/222,319

(22) Filed: Mar. 21, 2014

(51) Int. Cl. G01N 21/27; G01N 33/543; C12Q 1/68

(52) U.S. Cl. CPC G01N 21/27; C12Q 1/6825; G01N 33/54373; G01N 2201/061

(57) ABSTRACT

A sensor includes a multilayer dielectric having a nanohole array formed therein, where the multilayer dielectric has a base substrate layer and more than one dielectric layer formed on the base substrate layer. The multilayer dielectric is configured to support an optical Bloch surface state in response to a source light, and where the surface state is formed at an interface of the multilayer dielectric and an analyte. The nanohole array includes a plurality of nanoholes arranged in a periodic pattern, where the plurality of nanoholes extends at least partially through the multilayer dielectric.

### BACKGROUND

Surface states are electromagnetic states that may be formed at the surface (or interface) of two materials, for example between a metal and a dielectric, or between a dielectric and a photonic crystal. They are formed due to the transition of materials, and are found in layers of material closest to the interface. The materials forming the interface may include the air, a vacuum, a dielectric material, a metal, etc. The sharp termination of the first material leads to a change of the electromagnetic band structure from the first material to the second.

### SUMMARY

One embodiment relates to a sensor including a multilayer dielectric comprising a nanohole array formed therein. The multilayer dielectric has a base substrate layer and more than one dielectric layer formed on the base substrate layer. The multilayer dielectric is configured to support an optical Bloch surface state in response to a source light, and where the surface state is formed at an interface of the multilayer dielectric and an analyte. The nanohole array comprises a plurality of nanoholes arranged in a periodic pattern, where the plurality of nanoholes extends at least partially through the multilayer dielectric.

Another embodiment relates to a method of sensing a characteristic of an analyte. The method includes providing a multilayer dielectric having a nanohole array formed therein. The multilayer dielectric comprises a base substrate layer and more than one dielectric layer formed on the base substrate layer, where the multilayer dielectric is configured to support an optical Bloch surface state in response to a source light, and where the surface state is formed at an interface of the multilayer dielectric and the analyte. The nanohole array comprises a plurality of nanoholes arranged in a periodic pattern, where the plurality of nanoholes extends at least partially through the multilayer dielectric. The method further includes directing the source light at the multilayer dielectric such that the source light transmits through the nanohole array and the surface state is formed at the interface, and detecting and analyzing output based on the surface state and the analyte.

Another embodiment relates to a sensor system comprising a multilayer dielectric having a nanohole array formed therein. The multilayer dielectric comprises a base substrate layer and more than one dielectric layer formed on the base substrate layer, where the multilayer dielectric is configured to support an optical Bloch surface state in response to a source light, and where the surface state is formed at an interface of the multilayer dielectric and an analyte. The nanohole array comprises a plurality of nanoholes arranged in a periodic pattern, where the plurality of nanoholes extends at least partially through the multilayer dielectric. The system further comprises a light source configured to generate and direct the source light at the multilayer dielectric such that the source light transmits through the nanohole array and the surface state is formed at the interface, and a sensor configured to detect output of the nanohole array, and wherein the output is based on the surface state and the analyte.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIG. 1a is a block diagram of a multilayer nanohole array sensor according to one embodiment.

FIG. 1b is a block diagram of a sensor system according to one embodiment.

FIG. 2 is a schematic diagram of a multilayer nanohole array sensor according to one embodiment.

FIG. 3 is a schematic diagram of a multilayer nanohole array sensor according to one embodiment.

FIG. 4 is a schematic diagram of a multilayer nanohole array sensor according to one embodiment.

FIG. 5 is a schematic diagram of a multilayer nanohole array sensor according to one embodiment.

FIG. 6 is a schematic diagram of a sensor system according to one embodiment.

FIG. 7 is a schematic diagram of a pixelated nanohole array according to one embodiment.

FIG. 8 is a flowchart of a process for sensing using a multilayer nanohole array sensor according to one embodiment.

FIG. 9 is a flowchart of a process for sensing using a multilayer nanohole array sensor according to one embodiment.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the scope of the subject matter presented here.

Referring generally to the figures, various embodiments of multilayer nanohole sensors are shown and described. Surface states are bound electromagnetic states that are formed at a transition (e.g., an interface) of materials. Plasmons are one example of surface states which are formed for certain frequencies at the interface between a metal and a dielectric. Optical Bloch surface states are another example of a surface state which can be formed at an interface involving multi-layer dielectrics or photonic crystals; such surface states are discussed in Morozov et al., Surface EM Waves in 1D Photonic Crystals, arXiv:physics/0512272v1 (2005), and in Meade et al., Electromagnetic Bloch Waves at the Surface of a Photonic Crystal, Physical Review B, Vol. 44-19, 10961 (1991). One potential advantage of Bloch surface states over plasmons is that, because they involve dielectrics rather than metals, they can have less absorption losses than plasmons. Recently, a paper by Yanik et al., Seeing protein monolayers with naked eye through plasmonic Fano resonances, PNAS, Vol. 108-29, 11784 (2011), demonstrated a sensor using resonant transmission through an array of nanoholes in a metal, plasmon supporting, interface. The presence of an analyte at the interface causes a shift in the nanohole's resonance, shifting the transmission frequency. The sensitivity of the effect depends on the width of the resonance, which is limited by the absorption losses due to the finite resistivity of the metal.

In the embodiments described herein, the use of plasmons at a metal-analyte interface is replaced with Bloch surface states at an interface between a multilayer dielectric and the analyte. The multilayer dielectric is formed from more than one dielectric layer formed on a base substrate. The nanohole array is formed into the multilayer dielectric structure. When source light of a particular wavelength is propagated such that it is incident to the multilayer structure, it can produce surface states at the interface (i.e. optical Bloch surface states). When the structure contains a periodic array of nanoholes, a resonance can be induced to allow a certain portion of the light propagate through the nanohole array, as in the Yanik et al. device. However, because a dielectric structure is used in this embodiment, resistivity of the surface states is minimized, and resonances having narrower line widths are obtained, thereby allowing more sensitive detection of analytes. An analyte may be bound to the surface of the nanohole array (e.g., attached using a binding agent or applied within a microfluidic channel of the multilayer structure), and changes in transmissivity may be monitored to detect and measure various characteristics of the analyte. The analyte may be chemical in nature, either inorganic or organic. The analyte may also be biochemical in nature, for example, oligonuclide, protein, antibody, etc. The resonant frequency required for light propagation may also be monitored to detect and measure characteristics of the analyte. For example, absent the analyte it may be determined that a certain resonant frequency exists, but in the presence of the analyte, a different resonant frequency may exist. The difference in resonant frequencies may be analyzed to determine characteristics related to the analyte.

Typically, a dielectric is transparent (or semi-transparent) to certain bands of light. However, according to the embodiments described herein, by utilizing multiple layers of dielectric material, a multilayer dialectic structure may be formed such that it is reflective for a certain band of light (an “opaque band”). In some structures and frequency bands, this reflectivity may exist for all angles and polarizations; i.e., omnidirectional reflection as discussed in Fink et al., A Dielectric Omnidirectional Reflector, Science, Vol. 282, 1679 (1998). In other situations, the reflectivity may exist only for certain polarizations, or angular incidence ranges. When operating the sensor in such an opaque band, it will generally (i.e., absent the nanohole-induced resonances) have no transmission, despite being composed of dielectrics. This opacity allows the nanohole-induced resonant transmission to be readily observed without being masked by direct source transmission. When the multilayer dielectric structure is created, a nanohole array (consisting of a plurality of periodically spaced nanoholes) can be formed into the structure such that the resonance of the nanoholes is within the opaque band of the multilayer dielectric structure (i.e., the operating wavelength for the sensor created from the structure). In this manner, resulting multilayer dielectric structure can be opaque for light within the vicinity of the opaque band. However, when an analyte is bound to the surface of the structure (e.g., the nanoholes), the resonance of the nanoholes changes due to the presence of the analyte, and light may then transmit therethrough. The light that propagates through may then be spectrally resolved using a sensor (e.g. with a photosensor that is spectrally sensitive). By monitoring and analyzing data from the sensor, various characteristics related to the analyte may be obtained.

In one embodiment, the multilayer dielectric may be monitored to determine at what particular wavelength it is transparent (i.e. transmissive to light through the nanohole array). The multilayer dielectric is generally only transparent for a narrow frequency window corresponding to the resonant frequency of the nanoholes. This resonant frequency may shift dependent on a quantity of analyte bound to the surface of the multilayer dielectric. The amount of resonant frequency shift may be determined using a spectrally sensitive sensor, and this amount may be used to calculate the amount (or density) of the analyte, among other calculations.

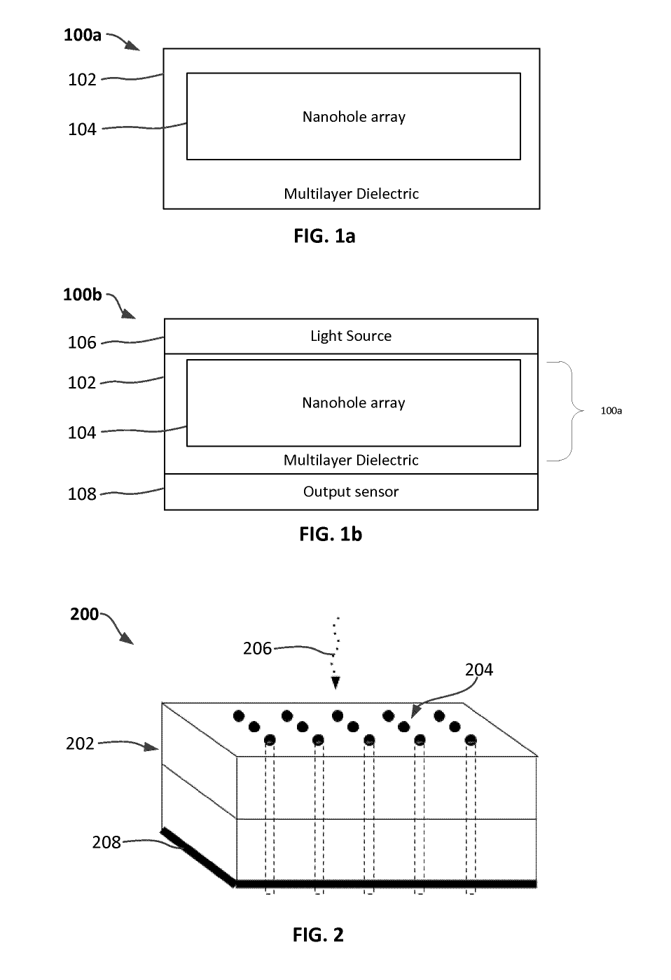

Referring to FIG. 1a, sensor 100 is shown according to one embodiment. Sensor 100a includes multilayer dielectric 102 and nanohole array 104. Multilayer dielectric 102 may include more than one dielectric layer formed on a base dielectric substrate. In one embodiment, multilayer dielectric 102 includes one or more metallic layers in addition to the more than one dielectric layer. In one embodiment, multilayer dielectric 102 includes one or more photonic crystals. Multilayer dielectric 102 has nanohole array 104 formed therein. Nanohole array 104 may be formed in multilayer dielectric 102 using any techniques known to those of skill in the art. Nanohole array 104 includes a plurality of nanoholes that extends at least partially through multilayer dielectric 102. Nanohole array 104 may be rectangular, square, hexagonal, circular, etc., in overall shape, and the plurality of nanoholes are arranged in the array in a periodic pattern (e.g., in a grid). Multilayer dielectric 102 is configured to support optical Bloch surface states to facilitate transmissive sensing of an analyte, which may be bound to a surface of multilayer dielectric 102 for analysis. As discussed above, the operating wavelength of sensor 100 may be within a spectral band for which multilayer dielectric 102 is opaque in the absence of nanohole array 104. Nanohole array 104 facilitates the transmission of a source light at the operating wavelength in order to induce surface states. Within the spectral band corresponding to the operating wavelength, multilayer dielectric 102 may function as an omnidirectional reflector or as a reflector where there is only nominal transmission. The reflectivity of multilayer dielectric 102 may be based on the incident angle or polarization of the source light disposed thereon. In one embodiment, the dielectric layers may be selected based on the characteristics of a source light (e.g., polarity and angle of incidence, etc.) in order to obtain desired reflectivity characteristics. In one embodiment, nanohole array 104 induces transmission of light through multilayer dielectric 102 for a resonant frequency of nanohole array 104, and the resonant frequency of nanohole array 104 may be based on the periodicity of the arrangement of the nanoholes.

Referring to FIG. 1b, a block diagram of sensor system 100b is shown according to one embodiment. System 100b includes sensor 100a comprising multilayer dielectric 102 and nanohole array 104. System 100b further includes light source 106 and output sensor 108. Light source 106 includes all components (e.g., lenses, polarizers, filters, control circuitry, etc.) necessary to generate and direct light at sensor 100a. In one embodiment, light source 106 includes a tunable laser. In another embodiment, light source 106 includes a broad spectrum light bulb and an optical filter configured to produce light of a desired wavelength. In another embodiment, light source 106 includes an LED and a filter to produce light of a desired spectrum. In general, the angle of incidence of the source light directed at sensor 100a may be set manually or it may be mechanically adjusted (e.g., using an actuator attached to light source 106 and a control circuit, etc.). Accordingly, light source 106 may be configured to direct light such that the light is normally incident or non-normally incident to sensor 100a. Source light may be directed in any direction or from any position with respected to sensor 100a. A polarizer may also be used to adjust the polarization of the source light as it is directed at sensor 100a. Output sensor 108 is generally configured to detect output from sensor 100a (e.g., light that has propagated through nanohole array 104). In one embodiment, output sensor 108 includes a photosensor that is spectrally sensitive. In this manner, sensing using sensor 100a may be facilitated by monitoring output sensor 108 (e.g., with a computing device) to detect an increase or decrease in the transmissivity of sensor 100a in the presence of an analyte. For example, an amount of a shift in a resonant frequency caused by the analyte near the surface of sensor 100a may be calculated using data provided by output sensor 108. The light source may be directed through the multilayer dielectric from the analyte side, or from the substrate side.

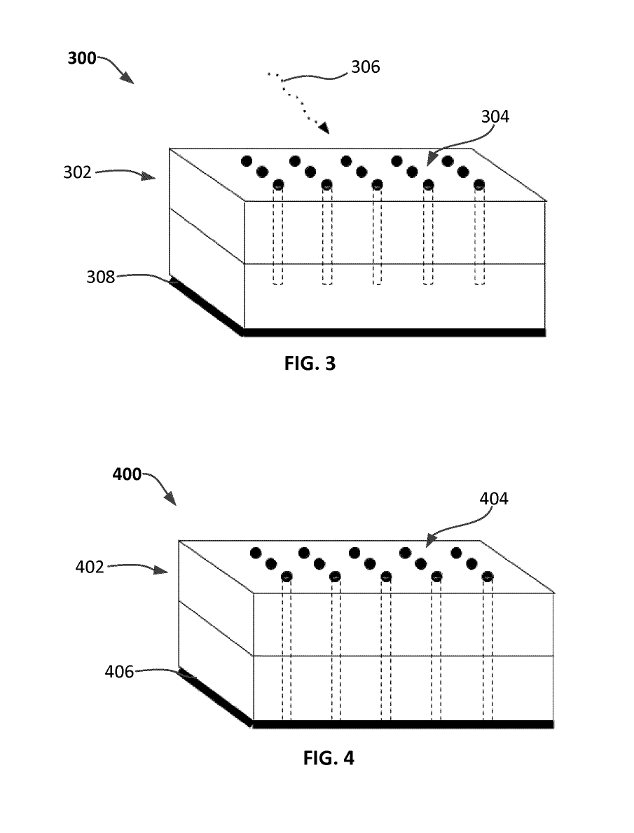

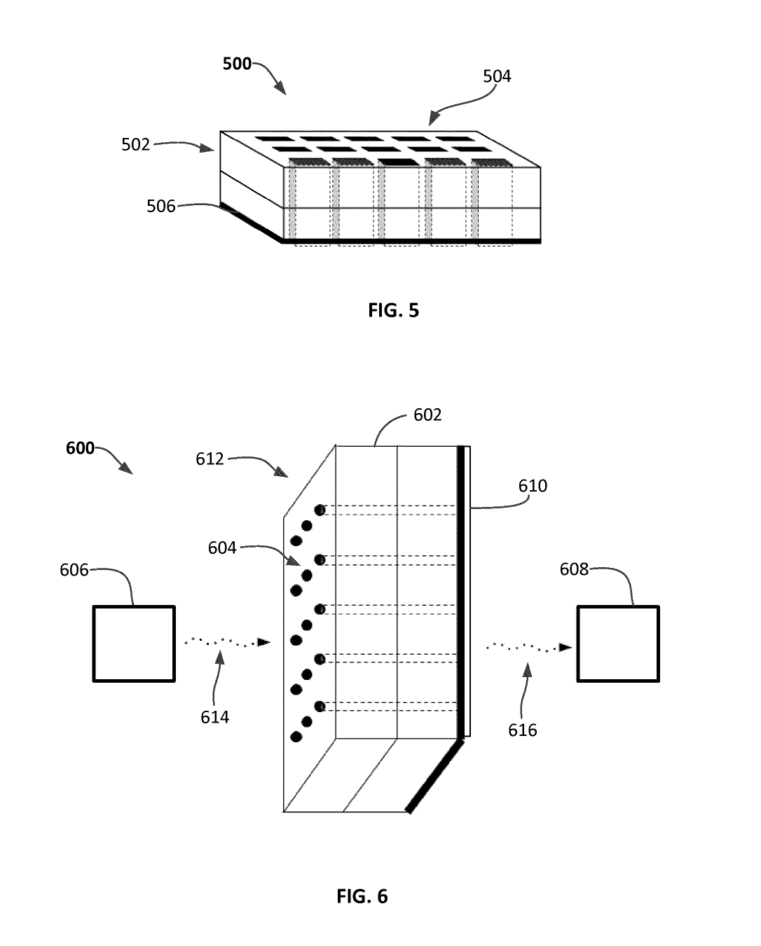

Referring to FIGS. 2-5, sensors 200, 300, 400, and 500 are shown according to various embodiments. Sensor 200 includes multilayer dielectric 202 having nanohole array 204 formed therethrough. In this embodiment, the nanoholes of nanohole array 204 are formed such that they extend entirely through multilayer dielectric 202, including base dielectric substrate layer 208. Source light 206 is depicted as being directed such that it is normal to the surface of sensor 200 (i.e. the surface of multilayer dielectric 202 having nanohole array 204).

Sensor 300 includes multilayer dielectric 302 having nanohole array 304 formed therethrough. In this embodiment, the nanoholes of nanohole array 304 are formed such that they extend only partially through multilayer dielectric 302 and stop within multilayer dielectric 302 without reaching base substrate layer 308. In another embodiment, nanohole array 304 may extend to any particular layer and stop within multilayer dielectric 302. Source light 306 is depicted as being directed such that it is off-normally incident to the surface of sensor 300 (i.e. the surface of multilayer dielectric 302 having nanohole array 304).

Other configurations of nanohole arrays are also envisioned. In one embodiment, sensor 400 includes multilayer dielectric 402 having nanohole array 404 formed therethrough. In this embodiment, the nanoholes of nanohole array 404 are formed such that they extend through multilayer dielectric 402 and terminate within base substrate dielectric layer 406. In another embodiment, sensor 500 includes multilayer dielectric 502 having nanohole array 504 formed therethrough. In this configuration, the nanoholes of nanohole array 504 are formed such that they extend through multilayer dielectric 502 and through base substrate dielectric layer 506. Additionally, the nanoholes of nanohole array 504 are depicted as having rectangular cross-sections. However, other cross-section configurations and combinations of cross-section configurations may be utilized. For example, in any of the embodiments discussed herein, the cross-sections of the nanoholes may be circular, elliptical, square, and slit-like with an elongated axis, etc. As another example, one embodiment includes a first portion of nanoholes that have a first cross-section configuration, and a second portion of nanoholes that have a second cross-section configuration that is different than the first. Further, the nanohole array may be an upward or downward feature in the multilayer dielectric (e.g., formed at the base dielectric substrate or formed at the surface dielectric layer).

Referring to FIG. 6, a schematic diagram of sensor system 600 is shown according to one embodiment. System 600 include sensor 612 which is comprised of multilayer dielectric 602, which has nanohole array 604 formed therethrough. System 600 further includes light source 606 (e.g., light source 106 in FIG. 1b). Light source 606 generates and directs source light beam 614 at the surface of sensor 612 such that the light is normal to the surface of sensor 612. Light source 606 may also be configured to direct source light beam 614 such that it is non-normal (e.g., diagonally incident) to the surface of sensor 612. Source light beam 614 is of wavelength within the opaque band for sensor 612 (i.e. multilayer dielectric 602 is opaque to light beam 614). Nanohole array 604 is configured such that frequency of resonance of induced surfaces states are within the opaque band when absent analyte 610. However, due to the presence of analyte 610, which may be bound to the surface of sensor 612, the resonant frequency is altered such that a portion of source light 614 may propagate through nanohole array 604. Any output light 616 that propagates through nanohole array 604 may be resolved using sensor 608 (e.g. a spectrally sensitive photosensor, etc.), and various characteristics of analyte 610 may be determined. As in other embodiments, the source light may be directed (as shown) from the analyte side or (not shown) from the substrate side.

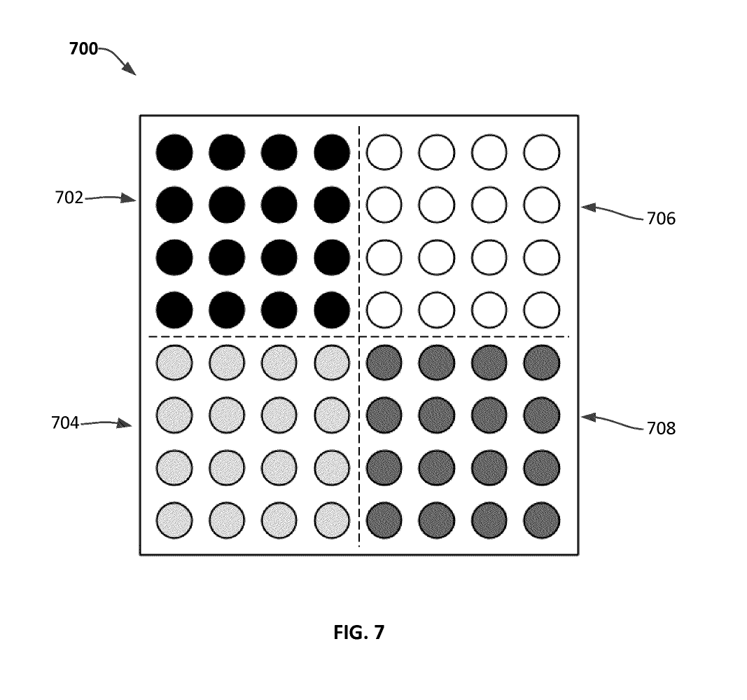

Referring to FIG. 7, a schematic diagram of pixelated nanohole array 700 is shown according to one embodiment. Pixelated nanohole array 700 is shown as having four pixels (pixel 702, pixel 704, pixel 706, and pixel 708) corresponding to four areas of the array. Each pixel is shown as corresponding to a 4×4 group of nanoholes. It should be noted that the scope of the present disclosure is not limited to a certain pixel arrangement or nanohole-per-pixel configuration. The pixelated arrangement allows for different pixels (or groups of pixels) to have one or more configurations (e.g., different nanohole depths, different nanohole cross-section types, different periodicities, different nanohole spacing, etc.). For example, certain pixels may be configured to be more sensitive than others. As another example, different binding agents may be applied to different pixels. In this manner, a sensor having pixelated nanohole array 700 may be used to detect different components of an analyte. For example, the pixels may be used to detect different molecules of an antibody solution analyte as the different binding agents of the pixels only bind to certain molecules of the analyte. Thus, certain pixels corresponding to a first binding agent may be used to analyze a first component of the analyte, whereas certain pixels corresponding to a second binding agent may be used to analyze a second component of the analyte.

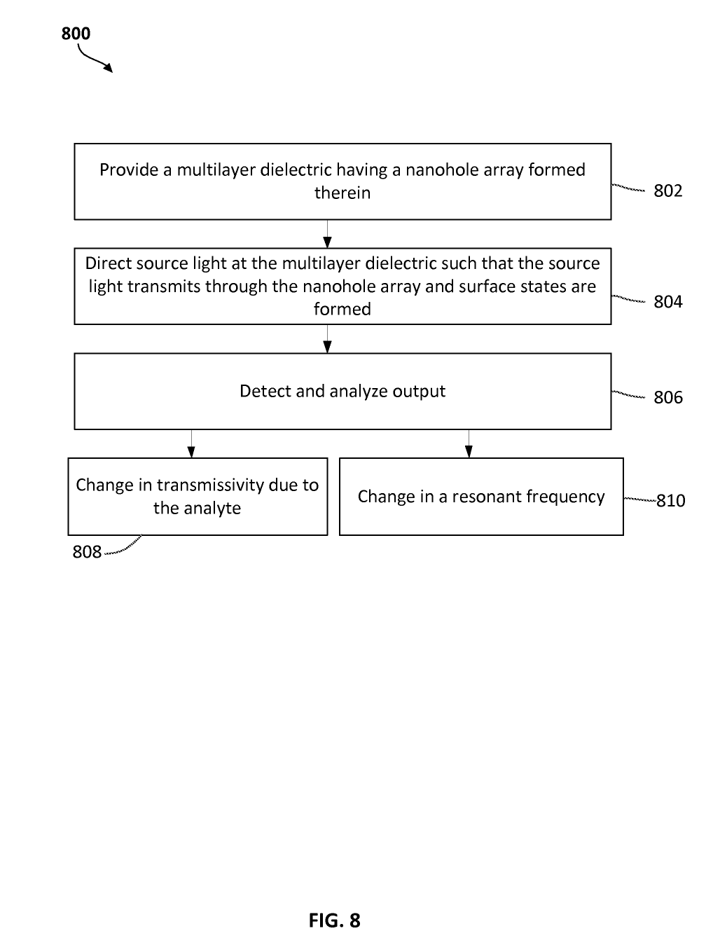

Referring to FIG. 8, a flow diagram of a process 800 for sensing using a multilayer nanohole array sensor is shown, according to one embodiment. In alternative embodiments, fewer, additional, and/or different actions may be performed. Also, the use of a flow diagram is not meant to be limiting with respect to the order of actions performed. A multilayer dielectric having a nanohole array formed therein (the sensor) is provided (802). The sensor has an analyte that is bound to its surface (e.g., using a binding agent). A source light is directed at the multilayer dielectric such that the source light transmits through the nanohole array (in the presence of an analyte) and surface states are formed at the interface of the sensor and the analyte (804). Source light may be directed (and/or the nanohole array sensor may be arranged) such that the source light arrives at the interface before the substrate, or such that the source light arrives at the substrate before the interface. Output from the sensor is detected and analyzed (806). For example, a spectrally sensitive photosensor may be used to detect such output. Analysis of the output may include determining a change in and amount of transmissivity due to the analyte at the interface (808) or determining a change in resonant frequency due to the analyte at the interface (810).

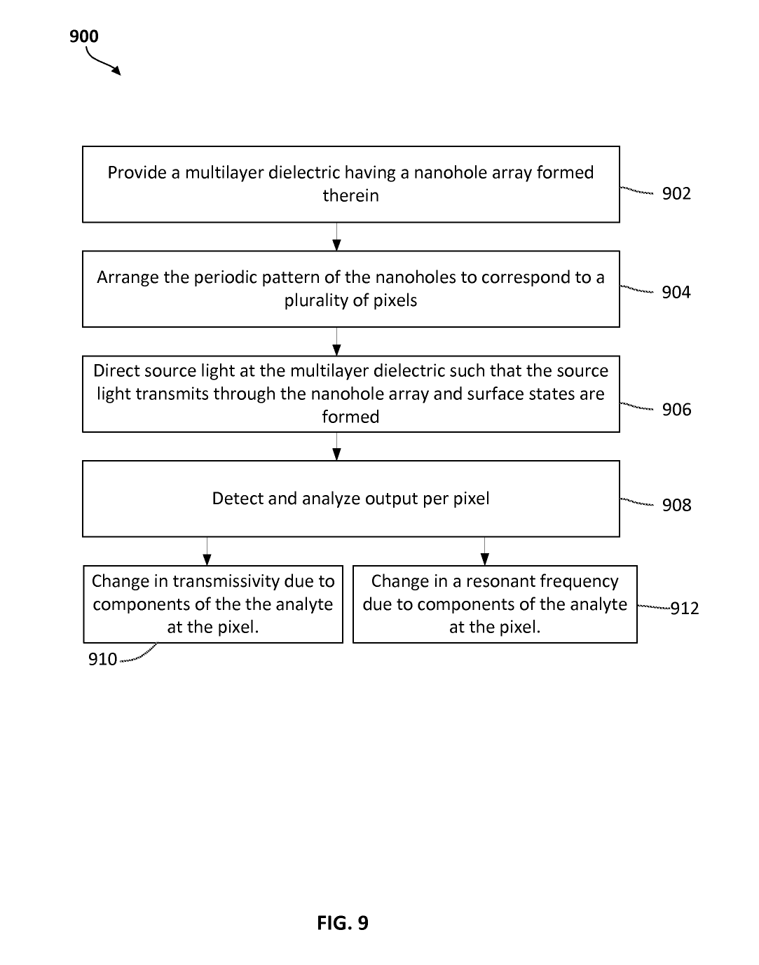

Referring to FIG. 9, a flow diagram of a process 900 for sensing using a multilayer nanohole array sensor is shown, according to one embodiment. In alternative embodiments, fewer, additional, and/or different actions may be performed. Also, the use of a flow diagram is not meant to be limiting with respect to the order of actions performed. A multilayer dielectric having a nanohole array formed therein (the sensor) is provided (902). The nanohole array has a periodic pattern that is configured to correspond to a plurality of pixels (904). As discussed above, the pixels may have differing sensitivities or may have differing binding agents disposed thereon in order to bind different components of an analyte. A source light is directed at the multilayer dielectric such that the source light transmits through the nanohole array (in the presence of an analyte) and surface states are formed at the interface of the sensor and the analyte (906). Output from the sensor is detected and analyzed (908). For example, a spectrally sensitive photosensor may be used to detect such output. Analysis of the output may include determining a change in and amount of transmissivity due to the components of analyte corresponding to a particular pixel (910) or determining a change in resonant frequency due to the components of analyte corresponding to a particular pixel (912).

The construction and arrangement of the systems and methods as shown in the various embodiments are illustrative only. Although only a few embodiments have been described in detail in this disclosure, many modifications are possible (e.g., variations in sizes, dimensions, structures, shapes and proportions of the various elements, values of parameters, mounting arrangements, use of materials, colors, orientations, etc.). For example, the position of elements may be reversed or otherwise varied and the nature or number of discrete elements or positions may be altered or varied. Accordingly, all such modifications are intended to be included within the scope of the present disclosure. The order or sequence of any process or method steps may be varied or re-sequenced according to alternative embodiments. Other substitutions, modifications, changes, and omissions may be made in the design, operating conditions and arrangement of the embodiments without departing from the scope of the present disclosure.

The present disclosure contemplates methods, systems and program products on any machine-readable media for accomplishing various operations. The embodiments of the present disclosure may be implemented using existing computer processors, or by a special purpose computer processor for an appropriate system, incorporated for this or another purpose, or by a hardwired system. Embodiments within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. When information is transferred or provided over a network or another communications connection (either hardwired, wireless, or a combination of hardwired or wireless) to a machine, the machine properly views the connection as a machine-readable medium. Thus, any such connection is properly termed a machine-readable medium. Combinations of the above are also included within the scope of machine-readable media. Machine-executable instructions include, for example, instructions and data which cause a general purpose computer, special purpose computer, or special purpose processing machines to perform a certain function or group of functions.

Although the figures may show a specific order of method steps, the order of the steps may differ from what is depicted. Also two or more steps may be performed concurrently or with partial concurrence. All such variations are within the scope of the disclosure. Likewise, software implementations could be accomplished with standard programming techniques with rule-based logic and other logic to accomplish the various connection steps, processing steps, comparison steps and decision steps.

While various aspects and embodiments have been disclosed herein, other aspects and embodiments will be apparent to those skilled in the art. The various aspects and embodiments disclosed herein are for purposes of illustration and are not intended to be limiting, with the true scope being indicated by the following claims.

1. A sensor, comprising:
a multilayer dielectric having a nanohole array formed therein, wherein the multilayer dielectric comprises a base substrate layer and more than one dielectric layer formed on the base substrate layer, wherein the multilayer dielectric is configured to support a Bloch surface state in response to a source light, wherein the surface state is formed at an interface of the multilayer dielectric and an analyte; and
wherein the nanohole array comprises a plurality of nanoholes arranged in a periodic pattern, and the plurality of nanoholes extends at least partially through the multilayer dielectric.

2. The sensor of claim 1, wherein an operating wavelength of the source light is within a spectral band for which the multilayer dielectric is at least substantially opaque absent the nanohole array.

3. The sensor of claim 2, wherein the spectral band is such that the multilayer dielectric functions as an omnidirectional reflector.

4. The sensor of claim 2, wherein reflectivity of the multilayer dielectric is based on a specified incident angle of the source light.

5. The sensor of claim 2, wherein reflectivity of the multilayer dielectric is based on a specified polarization of the source light.

6. The sensor of claim 1, wherein the nanohole array induces a transmission through the multilayer dielectric for a resonant frequency corresponding to the nanohole array.

7. The sensor of claim 6, wherein the resonant frequency is based on a periodicity of the periodic pattern of nanoholes.

8. The sensor of claim 1, wherein sensing using the multilayer dielectric is based on a change in a transmissivity due to the analyte at the interface.

9. The sensor of claim 1, wherein sensing using the multilayer dielectric is based on a change in a resonant frequency due to the analyte at the interface.

10. The sensor of claim 1, wherein the source light is normal to the multilayer dielectric.

11. The sensor of claim 1, wherein the source light is diagonal to the multilayer dielectric.

12. The sensor of claim 1, wherein the nanohole array extends through the entire multilayer dielectric.

13. The sensor of claim 1, wherein the nanohole array extends through the more than one dielectric layer and terminates within the base substrate layer.

14.-16. (canceled)

17. The sensor of claim 1, wherein each of the plurality of nanoholes has a cross-section that is at least one of circular, elliptical, rectangular, square, and slit-like with an elongated axis.

18. (canceled)

19. (canceled)

20. The sensor of claim 1, wherein the analyte is a biochemical.

21. The sensor of claim 20, wherein the biochemical is at least one of an oligonuclide, a protein, and an antibody.

22. (canceled)

23. The sensor of claim 1, wherein the sensor further comprises a binding agent disposed on the nanohole array, wherein the binding agent is based on the analyte.

24. (canceled)

25. (canceled)

26. The sensor of claim 1, wherein the more than one dielectric layer includes a metal layer.

27. (canceled)

28. (canceled)

29. The sensor of claim 1, wherein at least one of the pixels comprises a subset of the plurality of nanoholes having a unique nanohole configuration.

30. The sensor of claim 1, wherein the multilayer dielectric comprises a photonic crystal.

31. A method of sensing a characteristic of an analyte, comprising:
providing a multilayer dielectric having a nanohole array formed therein, wherein the multilayer dielectric comprises:
a base substrate layer and more than one dielectric layer formed on the base substrate layer, wherein the multilayer dielectric is configured to support a Bloch surface state in response to a source light, and wherein the surface state is formed at an interface of the multilayer dielectric and the analyte; and
wherein the nanohole array comprises a plurality of nanoholes arranged in a periodic pattern, and the plurality of nanoholes extends at least partially through the multilayer dielectric;
directing the source light at the multilayer dielectric such that the source light transmits through the nanohole array and the surface state is formed at the interface; and
detecting and analyzing output of the nanohole array based on the surface state and the analyte.

32. The method of claim 31, wherein the source light is directed at the interface before the substrate.

33. The method of claim 31, wherein the source light is directed at the substrate before the interface.

34. The method of claim 31, wherein an operating wavelength of the source light is within a spectral band for which the multilayer dielectric is at least substantially opaque when absent the nanohole array.

35.-39. (canceled)

40. The method of claim 31, wherein detecting and analyzing the output is based on a change in a transmissivity due to the analyte at the interface.

41. The method of claim 31, wherein detecting and analyzing the output is based on a change in a resonant frequency due to the analyte at the interface.

42.-54. (canceled)

55. The method of claim 31, wherein the nanohole array further comprises a binding agent, wherein the binding agent is based on the analyte.

56.-58. (canceled)

59. The method of claim 31, wherein the periodic pattern is such that the nanohole array represents a plurality of pixels, and wherein each of the pixels comprises a subset of the plurality of nanoholes, and wherein detecting and analyzing the output is based on output corresponding to a certain pixel.

60.-62. (canceled)

63. A sensor system, comprising:
a multilayer dielectric having a nanohole array formed therein, wherein the multilayer dielectric comprises:
a base substrate layer and more than one dielectric layer formed on the base substrate layer, wherein the multilayer dielectric is configured to support a Bloch surface state in response to a source light, wherein the surface state is formed at an interface of the multilayer dielectric and an analyte; and
wherein the nanohole array comprises a plurality of nanoholes arranged in a periodic pattern, and the plurality of nanoholes extends at least partially through the multilayer dielectric;
a light source configured to generate and direct the source light at the multilayer dielectric such that the source light transmits through the nanohole array and the surface state is formed at the interface; and
a sensor configured to detect output of the nanohole array, and wherein the output is based on the surface state and the analyte.

64. The system of claim 63, wherein the light source is configured to direct the source light at the interface before the substrate.

65. The system of claim 63, wherein the light source is configured to direct the source light at the substrate before the interface.

66. The system of claim 63, wherein an operating wavelength of the source light is within a spectral band for which the multilayer dielectric is at least substantially opaque absent the nanohole array.

67.-69. (canceled)

70. The system of claim 63, wherein the nanohole array induces a transmission through the multilayer dielectric for a resonant frequency corresponding to the nanohole array.

71. (canceled)

72. The system of claim 63, wherein sensing using the multilayer dielectric is based on a change in a transmissivity due to the analyte at the interface.

73. The system of claim 63, wherein sensing using the multilayer dielectric is based on a change in a resonant frequency due to the analyte at the interface.

74.-86. (canceled)

87. The system of claim 63, wherein the sensor further comprises a binding agent disposed on the nanohole array, wherein the binding agent is based on the analyte.

88.-90. (canceled)

91. The system of claim 63, wherein the periodic pattern is such that the nanohole array represents a plurality of pixels, and wherein each of the pixels comprises a subset of the plurality of nanoholes.

92.-94. (canceled)
